(19) United States
(12) Patent Application Publication
(10) Pub. No.: US 2023/0061584 A1
(43) Pub. Date: Mar. 2, 2023

(54) OPHTHALMIC SURGICAL INSTRUMENTS

(71) Applicant: ART, LIMITED, Grand Cayman (KY)

(72) Inventors: Ravi Nallakrishnan, Willowbrook, IL (US); Takayuki Akahoshi, Tokyo (JP)

(21) Appl. No.: 17/793,724

(22) PCT Filed: Jan. 27, 2021
(86) PCT No.: PCT/US21/15177

§ 371 Date: Jul. 19, 2022

Related U.S. Application Data
(60) Provisional application No. 62/966,104, filed on Jan. 27, 2020.

(51) Int. Cl. A61F 9/007

(52) U.S. Cl. CPC A61F 9/00763

(57) ABSTRACT

An ophthalmic surgical instrument for nucleus splitting includes an instrument handle having proximal and distal ends, wherein the distal end includes a pair of cooperating blade mounts which can be selectively moved relative to each other by manipulation of the handle. A pair of circular blade elements are respectively joined to the blade mounts at the distal end of the handle. Each of the blade elements have a lower cutting surface positioned generally beneath an axis defined by the respective blade mount. The lower cutting surfaces define a cutting edge for penetration of the nucleus of the eye when brought.

### FIELD OF THE INVENTION

The present invention relates generally to surgical instruments, and more particularly to ophthalmic surgical instruments having a pair of cooperating blade elements, which together define a cutting edge when placed in an abutting relationship, with the instruments being particularly configured to facilitate incision and splitting of the nucleus of a lens such as during a cataract removal procedure.

### BACKGROUND OF THE INVENTION

Phacoemulsification has come to be a technique of choice for the removal of damaged or diseased natural lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the apparatus itself is small and can fit through a relatively small incision, resulting in less fluid leakage from the eye capsule and shorter patient recovery times.

It is desirable to limit the amount of ultrasonic energy used as much as possible in order to minimize the risk of damage to eye tissue. Often, the lens nucleus (the hardest portion of the lens) is chopped or split into smaller pieces prior to or during phacoemulsification. Smaller pieces require less energy to emulsify, and this shortens the time during which ultrasonic energy is actually being supplied to the phacoemulsification apparatus.

Known fractionating techniques include making incisions into the lens and, thereafter, prying the incisions open to split the lens into halves or quarters. U.S. Pat. No. 6,262,682, hereby incorporated by reference in its entirety, discloses a surgical instrument which facilitates the chopping and splitting of the lens nucleus.

The present invention is directed to an improved construction for an ophthalmic surgical instrument which facilitates chopping and splitting of the lens nucleus, and separation of the nucleus fragments.

### SUMMARY OF THE INVENTION

In accordance with the present invention, an ophthalmic surgical instrument for nucleus splitting separation is disclosed which is particularly configured to facilitate chopping and splitting of a lens nucleus, and separation of the broken pieces of the nucleus. The instrument includes a handle having proximal and distal ends, wherein the distal end includes a pair of cooperating blade mounts which can be selectively moved relative to each other by manipulation of the handle.

The instrument includes a pair of blade elements respectively joined to the blade mounts at the distal end of the instrument handle. Each of the blade elements has at least a lower cutting surface positioned generally beneath an axis that is defined by the respective blade mount of each blade element.

In one form of the present invention, each of the blade elements has a generally circular shape and the lower cutting surfaces in a contacting relationship define a cutting edge for penetration of the nucleus of the eye.

In another form of the present invention, each of the blade elements includes an indicia therein in the form of either a recess or a through-hole. In one preferred form of the present invention, the indicia of each of the blade elements is circular in shape and is located substantially in the geometric center of the laterally-outwardly facing sides (exterior sides) of the blade elements

According to another aspect of the present invention, each of the blade elements has an upper cutting surface positioned generally above the axis of the respective blade mount such that the lower cutting surface and the upper cutting surface of each of the blade elements together extend substantially around a circumference of each of the blade elements.

According to yet another aspect of the present invention, each of the blade elements has a non-cutting, blunt surface positioned generally above the axis of the respective blade mount.

In another form of the present invention, each of the blade elements has a distal end free of any pointed cutting tip and each of the blade elements has a plurality of serrations formed on the lower cutting surface.

In another aspect of the present invention, the blade has a paddle shape wherein the lower cutting surfaces in a contacting relationship define a cutting edge for penetration of the nucleus. In one preferred form of the present invention, each of the blade elements includes a distal end in the form of a blunt edge connecting between the lower cutting surface and the non-cutting, blunt upper surface. In another preferred form of the present invention blunt edge at the distal end of each blade element is substantially flat. In yet another preferred form of the present invention, the blunt edge at the distal end of each blade element is semi-circular in shape. In another preferred form of the present invention, each blade element includes a sharpened, arcuate surface terminating at its distal end.

In accordance with another form of the present invention, an ophthalmic surgical instrument for nucleus splitting separation is disclosed which is particularly configured to facilitate chopping and splitting of a lens nucleus, and separation of the broken pieces of the nucleus. The instrument includes a handle having proximal and distal ends, wherein the distal end includes a pair of cooperating blade mounts which can be selectively moved relative to each other by manipulation of the handle.

The instrument includes a pair of blade elements respectively joined to the blade mounts at the distal end of the instrument handle. Each of the blade elements has at least a lower cutting surface positioned generally beneath an axis that is defined by the respective blade mount of each blade element and a non-cutting, blunt upper surface positioned generally above the axis. Each of the blade elements has a semi-circular shape, wherein the lower cutting surfaces terminate in a pointed distal end and in a contacting, relationship define a cutting edge for penetration of the nucleus. In one presently preferred form of the invention, one or both of the blade elements includes an indicia therein in the form of either a recess or a through-hole, the indicia having the form of a semi-circular shape with an arcuate side located adjacent the non-cutting, blunt upper surface and a flat side located adjacent the lower cutting surface.

In one presently preferred form of the invention, the handle of the present surgical instrument can be configured generally as forceps to activate and manipulate the cooperating blade elements of the instrument by either a regular action, or a reverse action. That is, a hand grip portion of the instrument handle can be configured such that it can be squeezed to move the blade elements toward each other (regular action) or can be configured such that the hand grip portion of can be squeezed to move the blade elements away from each other (reverse action.)

In one presently preferred form of the invention, the handle of the instrument includes a recessed, substantially flat portion aligned with a blunt edge of the blade elements.

Other features and advantages will become readily apparent from the accompanying drawings and the appended claims.

### BRIEF DESCRIPTION OF THE DRAWINGS

In the accompanying drawings forming part of the specification, in which like numerals are employed to designate like parts throughout the same,

FIG. 1 is a greatly enlarged, fragmentary, perspective view of the operative, distal portions of a first embodiment of an ophthalmic surgical instrument according to the present invention;

FIG. 2 is a fragmentary, side elevation view of the handle portions of the ophthalmic surgical instrument of FIG. 1;

FIG. 3 is a fragmentary, side elevation view of the blade portions of the ophthalmic surgical instrument of FIG. 1;

FIG. 4 is a fragmentary, front elevation view of the blade portions of the ophthalmic surgical instrument of FIG. 3, and FIG. 4 shows only the blunt edges of the blade portions;

FIG. 5 is a fragmentary, bottom plan view of the blade portions of the ophthalmic surgical instrument of FIG. 3, and FIG. 5 shows the cutting edges of the blade portions;

FIG. 6 is a fragmentary, side elevation view of the operative end of the ophthalmic surgical instrument of FIG. 1 in comparison to another ophthalmic surgical instrument;

FIG. 7 is a cross-sectional view of the handle portions of the ophthalmic surgical instrument taken along plane 7-7 in FIG. 6;

FIG. 8 is a fragmentary, cross-sectional view of the blade portions of the ophthalmic surgical instrument taken along plane 8-8 in FIG. 6;

FIG. 9 is a greatly enlarged, fragmentary, side elevation view of an operative portion of a second embodiment of an ophthalmic surgical instrument according to the present invention wherein only a distal portion of the instrument blade portions are illustrated in FIG. 9;

FIG. 10 is a fragmentary, top plan view of the blade portions of the ophthalmic surgical instrument of FIG. 9, and FIG. 10 shows the cutting edges of the blade portions extending around the majority of the perimeter of the blade portions;

FIG. 11 is a greatly enlarged, fragmentary, side elevation view of a portion of a third embodiment of an ophthalmic surgical instrument according to the present invention wherein only a fragmentary, distal portion of the instrument blade portions are illustrated in FIG. 11;

FIG. 12 is a greatly enlarged, fragmentary, side elevation view of a portion of a fourth embodiment of an ophthalmic surgical instrument according to the present invention wherein only a fragmentary, distal portion of the instrument blade portions are illustrated in FIG. 12;

FIG. 13 is a greatly enlarged, fragmentary, side elevation view of a portion of a fifth embodiment of an ophthalmic surgical instrument according to the present invention wherein only a fragmentary, distal portion of the instrument blade portions are illustrated in FIG. 13;

FIG. 14 is a greatly enlarged, fragmentary, side elevation view of a portion of a sixth embodiment of an ophthalmic surgical instrument according to the present invention wherein only a fragmentary, distal portion of the instrument blade portions are illustrated in FIG. 14;

FIG. 15 is a greatly enlarged, fragmentary, side elevation view of a portion of a seventh embodiment of an ophthalmic surgical instrument according to the present invention wherein only a fragmentary, distal portion of the instrument blade portions are illustrated in FIG. 15;

FIG. 16 is a greatly enlarged, fragmentary, perspective view of a portion of an eighth embodiment of an ophthalmic surgical instrument according to the present invention wherein only a fragmentary, distal portion of the instrument blade portions are illustrated in FIG. 16; and

FIG. 17 is a greatly enlarged, fragmentary, perspective view of a portion of a ninth embodiment of an ophthalmic surgical instrument according to the present invention wherein only a fragmentary, distal portion of the instrument blade portions are illustrated in FIG. 17.

### DETAILED DESCRIPTION OF THE PREFERRED EMBODIMENTS

While the present invention is susceptible of embodiment in various forms, there is shown in the drawings and will hereinafter be described the presently preferred embodiments, with the understanding that the present disclosure should be considered as an exemplification of the invention and is not intended to limit the invention to the specific embodiments illustrated.

In accordance with the illustrated embodiments, the present ophthalmic surgical instrument 10 comprises an instrument handle 12 having proximal and distal ends, wherein the distal end includes a pair of cooperating blade mounts 14 which can be selectively moved relative to each other by manipulation of a hand grip portion of the handle 12 located intermediate of the proximal and distal ends.

As will be further described, the instrument further includes a pair of blade elements 16, which are a mirror image of one another, and are respectively joined to the blade mounts 14 at the distal end of the instrument handle 12. Each of the blade elements 16 includes a lower cutting surface 18, positioned generally beneath an axis 19 defined by the respective blade mount 14, and non-cutting, blunt surface 20 positioned generally above the axis 19 of the respective blade mount 14. Each of the blade elements 16, includes an exterior surface 22 and an interior surface 24 facing or opposing the adjacent blade element 16. The exterior surfaces 22 of each blade element 16 may be substantially straight between the cutting surfaces 18 and the blunt surface 20 or may be convex or otherwise curved.

Notably, each of the cutting surfaces 18, when brought into a contacting, adjacent relationship, defines at least one, non-pointed or pointed, cutting surface or edge for penetration of the nucleus.

The handle 12 of the present surgical instrument 10 can be configured generally as forceps to activate and manipulate the cooperating blade elements 16 of the instrument by either a regular action, or a reverse action. That is, the hand grip portion of the instrument handle 12 can be configured such that it can be squeezed to move the blade elements 16 toward each other (regular action) or can be configured such that the hand grip portion of can be squeezed to move the blade elements away from each other (reverse action.)

The handle 12 of the instrument 10 can be made of lightweight titanium or medical grade stainless steel. The blade elements 16 of the instrument can be made of stainless titanium or steel of various grades, including 420SS, 304SS, and 17-4 pH or other suitable materials.

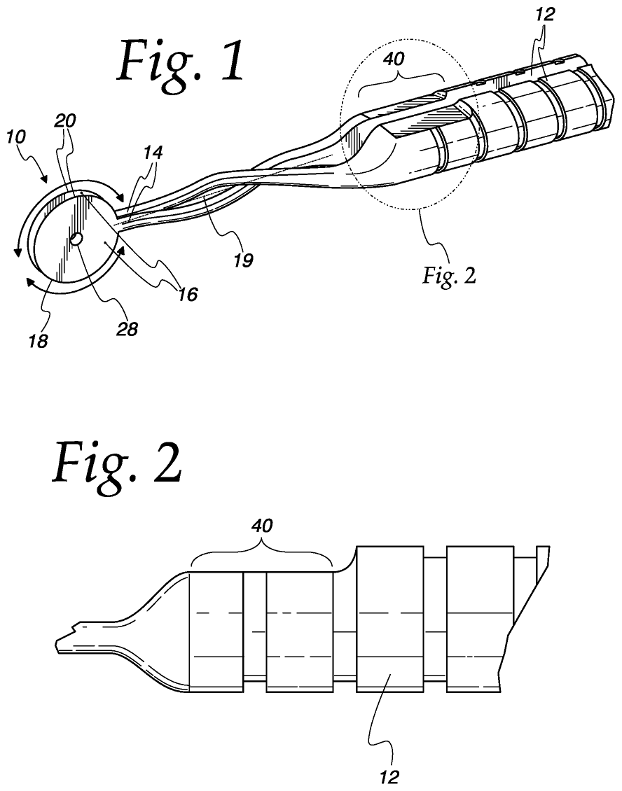

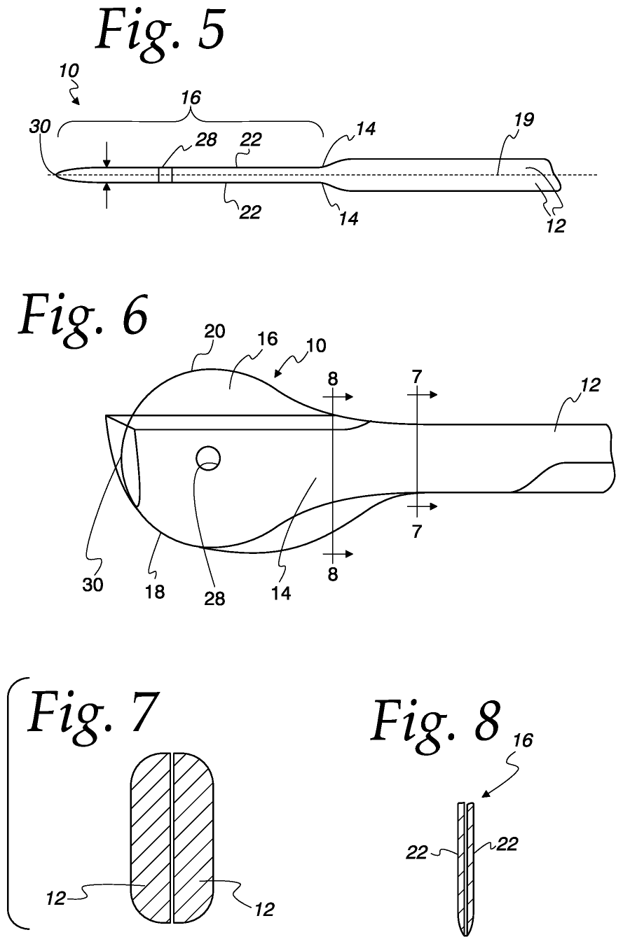

A first illustrated embodiment of a surgical instrument 10 according to the present invention is shown in FIGS. 1-8, wherein each of its blade elements 16 has a generally disc-like or circular shape that defines equal orthogonal axes 21, 23. Preferably, the axis 21 extends in a collinear or coextensive manner with the axis 19 of the blade mount 14 and handle 12. However, it will be appreciated that the axis 21 may be angled or offset from the axis 19 of the blade mount 14 for some applications.

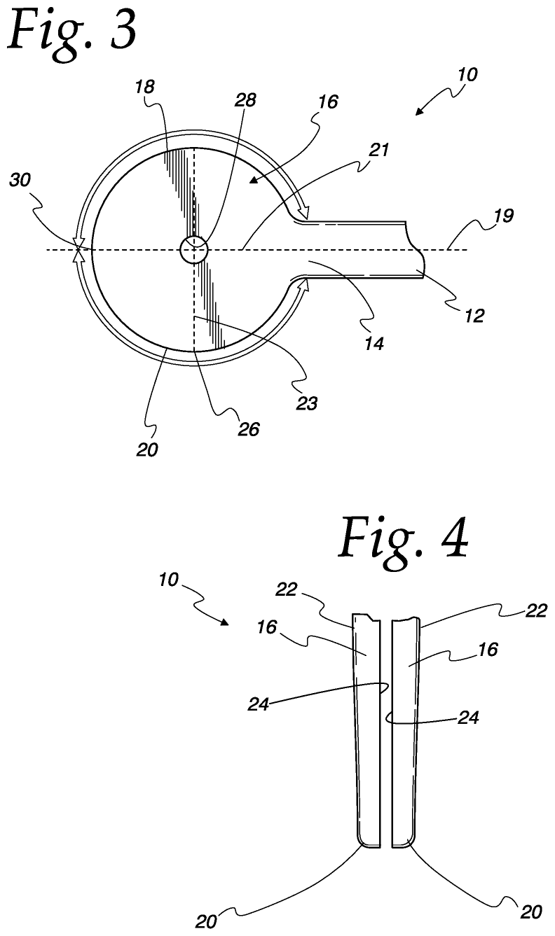

Referring to FIG. 3, the lower cutting surface 18 has a medial point 26, through which the axis 23 extends. Each lower cutting surface 18 extends from the blade mount 14 along one edge of the circular blade element 16 and terminates at a distal end 30 of the blade element 16, proximate to the axis 19. The blunt surface 20 extends along the opposite edge of the circular blade element 16 from the blade mount 14 to the distal end 30. When the blade elements 16 are brought together during operation by a surgeon, then the lower cutting surface 18 becomes a cutting edge in the form of a semi-circular arc. Likewise, when the blade elements 16 are brought together during operation by a user, then the upper blunt surface 20 has the form of a semi-circular arc.

In the illustrated first embodiment of the instrument 10, it is contemplated that each blade element 16 has a diameter (along the axes 21 and 23) on the order of 2.0 mm. The blade elements of the illustrated shapes can also be designed on micro handles, for movement through a micro-incision (i.e., less than about 1.0 mm.).

With reference to FIGS. 1 and 3, each blade element 16 includes an indicia 28 in the form of a generally circular through-hole located at the intersection of the axes 21, 23 and extending between the exterior surface 22 and the interior surface 24. The indicia 28 offers superior visualization within the surgical field for the surgeon to locate the lower cutting surface 18 and the upper blunt surface 20 during use of the instrument 10. In some forms, the indicia 28 may have the form of a recess or indentation (i.e., not a through hole) in one or both of the blade elements 16.

With reference to FIGS. 1 and 2, the handle 12 includes a substantially flat portion 40 located at the same axial location on the handle 12 as the upper blunt surface 20 to further aid the user in locating both the lower cutting surface 18 and the upper blunt surface 20 during use of the instrument 10. The flat portion 40 is preferably recessed relative to the remainder of the handle 12 to further aid the user of the instrument 10 in locating the flat portion 40 by feel alone, without requiring the user to look at the instrument 10.

Each blade element 16 preferably has a greater thickness proximate the blunt surface 20 and a reduced thickness at the lower cutting surface 18.

The inventor has found that the first illustrated embodiment of the instrument 10, having circular blade elements 16, is particularly advantageous and is most suitable for penetration into a hard grade nucleus with or without the support of a sustainer (not illustrated). The blunt surface 20 in the form of a semi-circular arc protects the posterior capsular bag from inadvertent rupture.

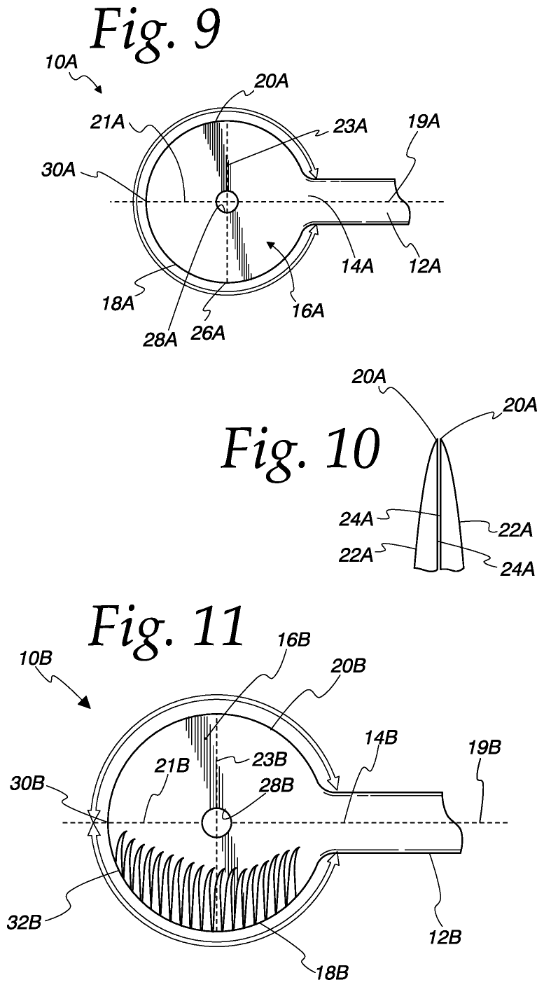

A second embodiment of a surgical instrument according to the present invention is shown in FIGS. 9 and 10, designated by the numeral 10A, and functions identically to the first illustrated embodiment of the instrument 10 as previously described. The numbered features of the second embodiment of the instrument 10A illustrated in FIGS. 9 and 10 are designated generally with the suffix letter “A” and are analogous to features of the first embodiment of the instrument 10 that share the same number (without the suffix letter “A”). The second embodiment of the surgical instrument 10A differs from the aforementioned first illustrated embodiment of the instrument 10 in that the second embodiment includes an indicia 28A in the form of a non-through hole recess in each blade element 16A and furthermore the upper surface 20A of each blade element 16A is sharpened such that the cutting edge of each blade element 16A extends around the perimeter of each blade element 16A. In other words, the lower cutting surface 18A extends from the blade mount 14A to the distal point 30A and continues as an upper cutting surface 20A instead of any blunt, non-cutting edge. The indicia 28A may have the form of a notch, recess, through hole, etching, coloring, or protrusion (not illustrated) on the exterior surface 22A of the blade element 16A. The indicium or indicia 28A may be omitted altogether. The second embodiment of the instrument 10A offers a more versatile cutting means as compared to the first illustrated embodiment 10, however without the additional protection to the capsular bag offered by one or more blunt edges.

A third embodiment of a surgical instrument according to the present invention is shown in FIG. 11, designated by the numeral 10B, and functions nearly identically to the first illustrated embodiment of the instrument 10 as previously described. The numbered features of the third embodiment of the instrument 10B illustrated in FIG. 11 are designated generally with the suffix letter “B” and are analogous to features of the first embodiment of the instrument 10 that share the same number (without the suffix letter “B”). The instrument 10B has a pair of blade elements 16B (only one of which is visible in FIG. 11) which also have a generally circular shape. However, the lower cutting surface 18B of each blade element 16B differs from the prior-discussed embodiments in that it includes a plurality of teeth, serrations, or semi-sharp edges 32B. The serrated edge 32B extends between the blade mount 14B to the distal point 30B of each blade element 16B. When the blade elements 16B are brought together during operation by a user, then the lower cutting surface 18B becomes a cutting edge in the form of a serrated arc or semicircle. When the blade elements 16B are brought together during operation by the user, then the upper blunt surface 20B has the form of an arc or semi-circle.

A fourth embodiment of a surgical instrument according to the present invention is shown in FIG. 12, designated by the numeral 10C, and functions similarly to the first illustrated embodiment of the instrument 10 as previously described. The numbered features of the fourth embodiment of the instrument 10C illustrated in FIG. 12 are designated generally with the suffix letter “C” and are analogous to features of the first embodiment of the instrument 10 that share the same number (without the suffix letter

With reference to FIG. 12, each blade element 16C (only one of which is visible in FIG. 12) of the fourth illustrated embodiment of the instrument 10C is substantially paddle-shaped or rectangular and includes a lower, sharpened cutting surface 18C having a linear configuration, positioned beneath the axis 19C of the respective blade mount 14C, and an upper, blunt surface 20C having a linear configuration, positioned above the axis 19C. The lower cutting surface 18C of each blade element 16C terminates in a distal end 30C in the form of a flat, blunt edge 31C. In this embodiment of the instrument 10C, the operative, distal end of each blade element 16C is blunt, including radiused corners where the blunt edge 31C merges with the upper, blunt surface 20C and the lower, cutting surface 18C to provide additional protection against inadvertent posterior capsule rupture. The indicia 28C has the form of an enlarged through hole in each of the blade elements 16C.

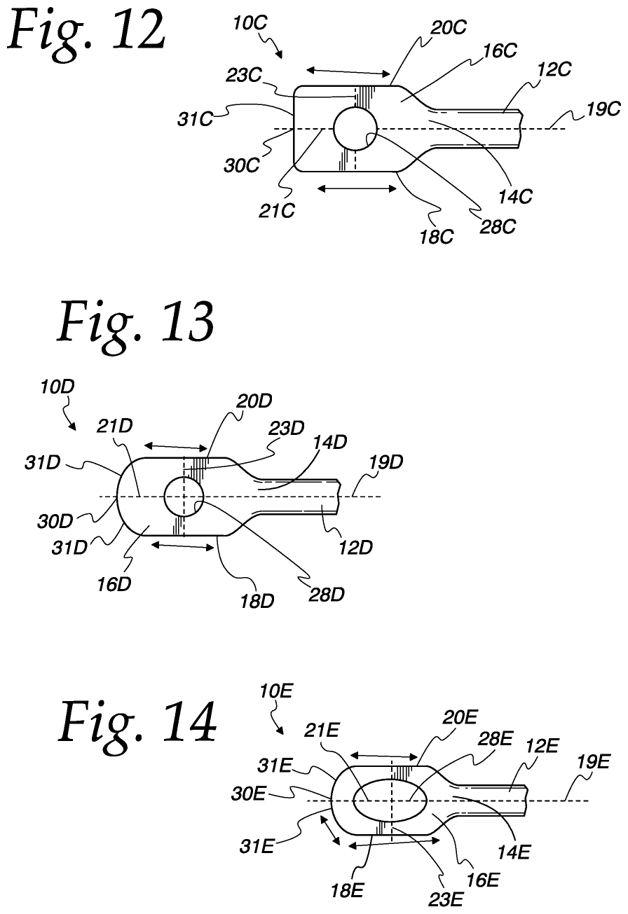

A fifth embodiment of a surgical instrument according to the present invention is shown in FIG. 13, designated by the numeral 10D, and functions similarly to the fourth illustrated embodiment of the instrument 10C as previously described. The numbered features of the fifth embodiment of the instrument 10D illustrated in FIG. 13 are designated generally with the suffix letter “D” and are analogous to features of the fourth embodiment of the instrument 10C that share the same number (without the suffix letter “C”).

Referring now to FIG. 13, each blade element 16D includes a lower, sharpened cutting surface 18D having a linear configuration, positioned generally beneath the axis 19D of the respective blade mount 14D. Each blade 16D includes a generally linear blunt surface 20D positioned above axis 19D of the respective blade mount 14D. The lower cutting surface 18D and blunt, upper surface 20D merge at a distal end 30D positioned on an arcuate, blunt surface 31D.

A sixth embodiment of a surgical instrument according to the present invention is shown in FIG. 14, designated by the numeral 10E, and functions similarly to the fifth illustrated embodiment of the instrument 10D as previously described. The numbered features of the sixth embodiment of the instrument 10E illustrated in FIG. 14 are designated generally with the suffix letter “E” and are analogous to features of the fifth embodiment of the instrument 10D that share the same number (without the suffix letter “E”).

Referring now to FIG. 14, each blade element 16E includes a lower, sharpened cutting surface 18E having a linear configuration, positioned generally beneath the axis 19E of the respective blade mount 14E. Each blade 16E includes a generally linear blunt surface 20E positioned above axis 19D of the respective blade mount 14E. The lower cutting surface 18E merges to the distal end 30E in a sharpened, arcuate surface 31E. The indicia 28E has the form of an elongate aperture or oval through hole in each of the blade elements 16E.

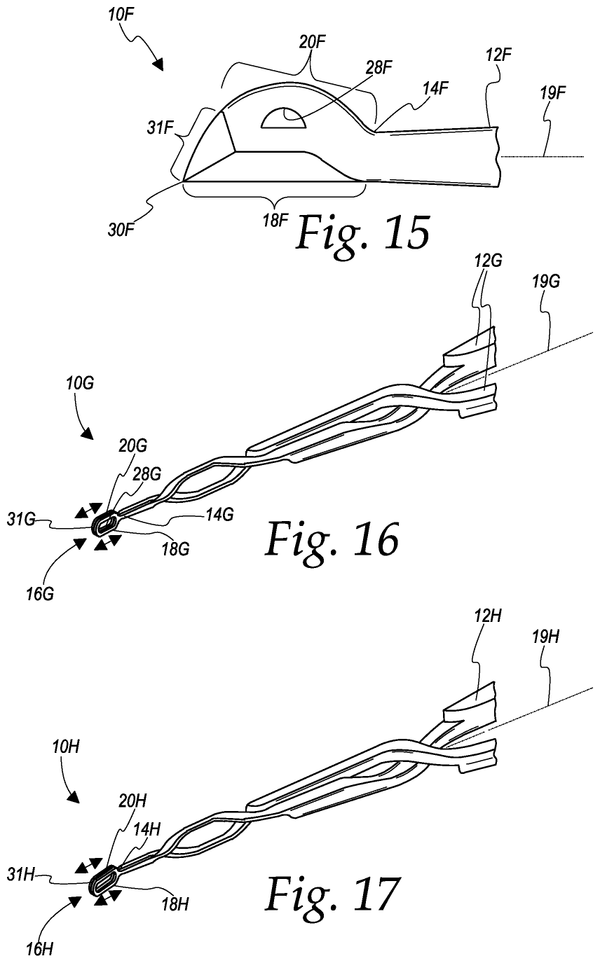

A seventh embodiment of a surgical instrument according to the present invention is shown in FIG. 15, designated by the numeral 10F, and functions similarly to the first illustrated embodiment of the instrument 10 as previously described. The numbered features of the seventh embodiment of the instrument 10 illustrated in FIG. 15 are designated generally with the suffix letter “F” and are analogous to features of the first embodiment of the instrument 10 that share the same number (without the suffix letter “F”).

Referring now to FIG. 15, each blade element 16F includes a lower, sharpened cutting surface 18F having a linear configuration, positioned generally beneath the axis 19F of the respective blade mount 14F. Each blade 16F includes a generally arcuate blunt surface 20F positioned above axis 19F of the respective blade mount 14F. The lower cutting surface 18F merges to its distal end 30F in a sharpened, arcuate surface 31F which functions as a secondary cutting surface. The indicia 28F has the form of a semi-circular through hole or recess in one or both of the blade elements 16F, which indicates to the user where the lower cutting surface 18F and the upper, blunt surface 20F are located on the instrument 10F. The inventor has found that the instrument 10F, having semi-circular shaped blade elements 16F which terminate in a cutting tip 40F at the distal ends 30F thereof, is especially suitable for a Grade 2+/3−nucleus, which may present a particularly difficult problem for prior art pre-chopping instruments. Such a nucleus is typically too hard to pre-chop using some prior art instruments, and yet such a nucleus is not hard enough for other prior art pre-chopping instruments.

An eighth embodiment of a surgical instrument according to the present invention is shown in FIG. 16, designated by the numeral 10G, and functions similarly to the fifth illustrated embodiment of the instrument 10D as previously described. The numbered features of the eighth embodiment of the instrument 10G illustrated in FIG. 16 are designated generally with the suffix letter “G” and are analogous to features of the fifth embodiment of the instrument 10D that share the same number (without the suffix letter “G”).

Referring now to FIG. 16, each blade element 16G of the instrument 10G includes a lower, sharpened cutting surface 18G having a linear configuration, positioned generally beneath the axis 19G of the respective blade mount 14G. Each blade 16G includes a generally linear blunt surface 20G positioned above axis 19G of the respective blade mount 14G. The lower cutting surface 18G merges to the distal end 30E in a blunt, arcuate surface 31G. The indicia 28G has the form of an enlarged, somewhat circular through hole in each of the blade elements 16G.

A ninth embodiment of a surgical instrument according to the present invention is shown in FIG. 17, designated by the numeral 10H, and functions similarly to the eighth illustrated embodiment of the instrument 10G as previously described. The numbered features of the ninth embodiment of the instrument 10H illustrated in FIG. 17 are designated generally with the suffix letter “H” and are analogous to features of the eighth embodiment of the instrument 10G that share the same number (without the suffix letter “H”).

Referring now to FIG. 17, each blade element 16H of the instrument 10H includes a lower, sharpened cutting surface 18H having a linear configuration, positioned generally beneath the axis 19H of the respective blade mount 14H. Each blade 16H includes a generally linear blunt surface 20H positioned above axis 19H of the respective blade mount 14H. The lower cutting surface 18H merges to the distal end 30H in a blunt, arcuate surface 31H. The indicia 28H has the form of an elongate aperture or oval through hole in each of the blade elements 16H.

The surgical instruments 10-10H are believed to possess an improved performance over one or more pre-choppers of the prior art in one or more of the following categories: insertion into the eye, separation of the nucleus, rotation and maneuverability of the instrument within the eye, and posterior capsule safety when separating the nucleus.

From the foregoing, it will be observed that numerous modifications and variations can be effected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

1. An ophthalmic surgical instrument for nucleus splitting, comprising:
an instrument handle having proximal and distal ends, wherein the distal end includes a pair of cooperating blade mounts which can be selectively moved relative to each other, and
a pair of blade elements respectively joined to said blade mounts at the distal end of said instrument handle,
each of said blade elements having at least a lower cutting surface positioned generally beneath an axis defined by the respective blade mount,
wherein each of said blade elements has a generally circular shape, and wherein said lower cutting surfaces in a contacting relationship define a cutting edge for penetration of the nucleus.

2. The ophthalmic surgical instrument for nucleus splitting in accordance with claim 1, wherein each of said blade elements includes an indicia therein in the form of either a recess or a through-hole.

3. The ophthalmic surgical instrument for nucleus splitting in accordance with claim 2, wherein said indicia of each of said blade elements is circular in shape and is located substantially in the geometric center of the laterally-outwardly facing sides of said blade elements.

4. The ophthalmic surgical instrument for nucleus splitting in accordance with claim 1, wherein each of said blade elements includes an upper cutting surface positioned generally above said axis.

5. The ophthalmic surgical instrument for nucleus splitting in accordance with claim 4, wherein said lower cutting surface and said upper cutting surface of each of said blade elements together extend substantially around a circumference of each of said blade elements.

6. The ophthalmic surgical instrument for nucleus splitting in accordance with claim 1, wherein each of said blade elements includes a non-cutting, blunt upper surface positioned generally above said axis.

7. The ophthalmic surgical instrument for nucleus splitting in accordance with claim 1, wherein each of said blade elements has a distal end, and wherein each of said blade elements has a plurality of serrations formed on said lower cutting surface.

8. The ophthalmic surgical instrument for nucleus splitting in accordance with claim 1, wherein each of said blade elements terminates at a distal end that is free of any pointed cutting tip.

9. An ophthalmic surgical instrument for nucleus splitting, comprising:
an instrument handle having proximal and distal ends, wherein the distal end includes a pair of cooperating blade mounts which can be selectively moved relative to each other, and
a pair of blade elements respectively joined to said blade mounts at the distal end of said instrument handle,
each of said blade elements having at least a lower cutting surface positioned generally beneath an axis C, defined by the respective blade mount,
each of said blade elements includes a non-cutting, blunt upper surface positioned generally above said axis,
wherein each of said blade elements has a paddle shape, and wherein said lower cutting surfaces in a contacting relationship define a cutting edge for penetration of the nucleus.

10. The ophthalmic surgical instrument for nucleus splitting in accordance with claim 9, wherein each of said blade elements includes a distal end in the form of a blunt edge connecting between said lower cutting surface and said non-cutting, blunt upper surface.

11. The ophthalmic surgical instrument for nucleus splitting in accordance with claim 10, wherein said blunt edge of each blade element is substantially flat.

12. The ophthalmic surgical instrument for nucleus splitting in accordance with claim 10, wherein said blunt edge of each blade element is semi-circular in shape.

13. The ophthalmic surgical instrument for nucleus splitting in accordance with claim 9, wherein each blade element includes a sharpened, arcuate surface terminating at a distal end.

14. An ophthalmic surgical instrument for nucleus splitting, comprising:
an instrument handle having proximal and distal ends, wherein the distal end includes a pair of cooperating blade mounts which can be selectively moved relative to each other, and
a pair of blade elements respectively joined to said blade mounts at the distal end of said instrument handle,
each of said blade elements having at least a lower cutting surface positioned generally beneath an axis defined by the respective blade mount,
each of said blade elements includes a non-cutting, blunt upper surface positioned generally above said axis,
wherein each of said blade elements has a semi-circular shape, and wherein said lower cutting surfaces terminate in a pointed distal end and in a contacting relationship define a cutting edge for penetration of the nucleus, and
wherein at least one of said blade elements includes an indicia therein in the form of either a recess or a through-hole, said indicia having the form of a semi-circular shape with an arcuate side located adjacent said non-cutting, blunt upper surface and a flat side located adjacent said lower cutting surface.

15. The ophthalmic surgical instrument for nucleus splitting in accordance with claim 1 wherein said instrument handle includes a recessed, substantially flat portion aligned with a blunt edge of said blade elements.

16. The ophthalmic surgical instrument for nucleus splitting in accordance with claim 1 wherein said instrument handle can be squeezed to move said blade elements together.

17. The ophthalmic surgical instrument for nucleus splitting in accordance with claim 1 wherein said instrument handle can be squeezed to move said blade elements apart.
